(19) United States
(12) Patent Application Publication
(10) Pub. No.: US 2013/0202788 A1
(43) Pub. Date: Aug. 8, 2013

(54) NON-AQUEOUS THIXOTROPIC FRAGRANCE GEL

(71) Applicant: The Dial Corporation, Scottsdale, AZ (US)

(72) Inventors: Tyler R. Mikkelsen, Scottsdale, AZ (US); Kevin Hafer, Scottsdale, AZ (US); Rosanne Benavides, Phoenix, AZ (US)

(73) Assignee: The Dial Corporation, Scottsdale, AZ (US)

(21) Appl. No.: 13/677,391

(22) Filed: Nov. 15, 2012

Related U.S. Application Data
(60) Provisional application No. 61/563,070, filed on Nov. 23, 2011.

(51) Int. Cl. A61L 9/04

(52) U.S. Cl. CPC A61L 9/048
USPC 427/212; 424/76.4; 366/348

(57) ABSTRACT

The present invention is a non-aqueous self-standing rigid fragrance gel comprising fragrance oil and a hydroxylalkyl cellulose derivative. In the preferred embodiment of the present invention, about 4.0 weight percent or more of hydroxylpropyl cellulose converts fragrance oil into a rigid gel with pseudoplastic rheology. The pseudoplastic gel shears to allow coating of crystals and objects, with subsequent rigidity restored to at least 80%.

### FIELD OF INVENTION

The present invention relates to an air freshener and in particular to a non-aqueous thixotropic fragrance gel that has free-standing structure.

### BACKGROUND OF THE INVENTION

Gel air fresheners have existed in the market and in the patent literature for decades. Such products may be aqueous and may comprise such few ingredients as a gelling/gellation agent (also referred to as a “gellant”), a fragrance oil, and water. The gelling agent may comprise a polymeric material such as naturally marine-sourced carrageenan, agar, or alginate, or it may comprise a complex blend of materials including carrageenan, a natural gum from non-marine sources, a cellulosic material, and various mono- and divalent cations, used together in various combinations to ensure rigidity of the solidified gel and its stability over time against syneresis. An exemplary gel air freshener product based on carrageenan natural gelling agent is Renuzit® Adjustables® Cone Air Freshener marketed by Henkel. Preferred aqueous gel air fresheners are rigid and “self-supporting.” These products look like solids, although closer inspection reveals they are rigid water gels having an outer surface that is easily penetrated during the procedure used to measure gel strength.

Additionally, nonaqueous solid to semi-solid air fresheners comprising fragrance dispersed within a polymer matrix are known. For example, a fragrance may be dispersed within a thermoset or thermoplastic polymer matrix. In the former case, a mixture of two components, such as epoxy resin and catalyst, are mixed together along with fragrance, and the resulting mixture is allowed to polymerize to form a plastic-to-rubbery scented article. In the latter case, a thermoplastic polymer is melted, often from a beaded polymer, to form a polymer melt. Fragrance is added to the hot melt and the mixture is cast into molds and cooled. A similar plastic-to-rubbery feel article is produce. In either case, thermosetting or thermoplastic, the resulting article has very intense initial fragrance strength, but that fragrance strength rapidly diminishes after just a few days exposure to ambient air. Furthermore, these fragranced polymer articles can only scent a very small space, such as the interior of a car or closet. These performance issues relate from one or more of the following technical issues: (1) There is an upper limit as to how much fragrance oil can be incorporated into a polymer matrix yet still have a solid object; (2) adding volatile fragrance oil to a heated polymer melt, or to ingredients that will generate heat upon polymerizing, will result in some flashing off of some of the fragrance; and, (3) fragrance is literally “trapped” within the polymer matrix with no easy way to volatize out into the environment at ambient temperatures.

Lastly, some is known about thickened fragrance oils. For example, fragrance oils are occasionally thickened to particular viscosity levels in order to better dispense from wick-based evaporative devices or to reduce aspiration hazard.

Some of the more relevant aqueous gel air freshener art is as follows:

U.S. Pat. No. 2,691,615 (1954, Turner, et al.) is a very early reference claiming a gel based air freshener. The reference discloses the use of agar-agar, gelatin, pectin, starch, and various gums as potential gelling agents for forming air conditioning gels. The aqueous air treating gel comprised of volatile air treatment compounds, water, and 1 to 4% of an aqueous gelling agent, (preferably agar-agar or calcium alginate), was found to be firm and “substantially devoid of syneresis.”

U.S. Pat. No. 2,927,055 (1960, Lanzet) discloses an air-treating gel comprising water, a volatile air treatment component, and a gelling agent mixture comprising carrageenan, Locust Bean gum, potassium chloride, and sodium carboxymethyl cellulose. The mixture is blended at around 170° F., then poured into molds and cooled. The inventors successfully balanced the amounts of these components to improve the viscosity/handling of the gel in the hot/molten state and to optimize stability, firmness, and appearance of the solidified gel.

U.S. Pat. No. 4,056,612 (1977, Lin) discloses an air freshener gel that utilizes a gelling agent mixture comprising carrageenan (mostly kappa and lambda), Locust Bean gum, and an ammonium salt. The inventive gels exhibited high water gel strengths and syneresis rates of less than 0.3%.

U.S. Pat. No. 4,178,264 (1979, Streit, et al.) discloses an improved air-treating gel composition comprising both carrageenan and a stearate salt used in combination as the gelling agent, wherein the preferred ratio of carrageenan to stearate is from about 0.3:1 to about 5:1. In addition to carrageenan, stearate, water, and volatile actives, a stearate solubility enhancer, such as a solvent or one of a variety of nonionic materials, to increase the solubility of the stearate in the aqueous environment. The preferred components for enhancing the stearate solubility include ethylene glycol, propylene glycol, and ethanol. Most of the Streit example compositions comprise propylene glycol, carrageenan, and sodium stearate combinations for rigid and stable gels.

U.S. Pat. No. 4,318,746 (1982, Claffey, et al) discloses a gel having improved physical stability that comprises the combination of a first polymer that dissolves, disperses or hydrates in hot water, and a second polymer that is insoluble in hot water. Claffey discloses that the first polymer is preferably carrageenan and that the second polymer is preferably a cellulose derivative that exhibits reversible thermal gelation properties in water. Such cellulose polymers include methyl cellulose, hydroxypropyl cellulose, hydroxypropyl methylcellulose, and hydroxybutyl methylcellulose.

U.S. Pat. No. 4,666,671 (1987, Purzycki, et al.) discloses fragranced gel blocks useful for deodorizing urinals and toilet bowls. These gel blocks comprise a gelling agent selected from fatty acid salts, sodium alginate, carboxymethyl cellulose, carrageenan, hydroxypropyl cellulose, starches, and gums, although the most preferred gelling agent disclosed is sodium stearate used alone. Solvents including lower alkyl alcohols, diols, and glycol ethers are optionally added to adjust the final melting temperature range of the gel block.

U.S. Pat. No. 5,643,866 (1997, Ansari, et al.) discloses an air-treating gel comprising dibenzylidene sorbitol acetal (DBSA) in combination with a glycol component as the aqueous gelling agent mixture. Such air freshener gels comprising fragrance, water, DBSA and glycol are shaped solid gel products that can withstand temperatures up to 50°-60° C. without melting.

U.S. Pat. No. 5,698,188 (1997, Evans) discloses a gel air fragrancing composition comprising carrageenan in accordance with Lanzet '055. The preferred compositions of Evans comprise 1-20% fragrance, 2-10% carrageenan constituent, and optional preservative and coloring agents, with the balance being water. The carrageenan constituents include commercial thickeners based on carrageenan that are likely to also include proprietary amounts of other materials such as Locust Bean gum, cellulose materials and calcium and/or potassium salts.

U.S. Pat. No. 5,741,482 (1998, Modi) discloses an air treatment gel composition comprising a seaweed gum, a guar or guar derivative, and an additional non-sulfonated water-soluble polymer selected from a group of numerous cellulose polymers.

Lastly, U.S. Patent Application Publication 2008/0317683 (2008, Trudso) discloses carrageenan compositions and products containing these compositions. The disclosure is directed to methods for extracting and producing carrageenans having a mixture of counter-ions (sodium, potassium, calcium and magnesium), wherein the carrageenan composition has a gelling temperature of between 7° C. and 30° C. An air freshener gel is disclosed that comprises the carrageenan composition having the optimized mixture of cations.

Some of the more relevant art related to scented polymers is as follows:

PCT Application Publication WO 00/26285 (Healy, et al.) discloses gelled esters obtained by mixing a suitable ester with one or more triblock copolymers, star polymers, radial polymers, multi-block copolymers, or mixtures thereof. Optionally, one or more diblock copolymers can be used in forming the gel compositions.

U.S. Pat. No. 3,685,734 (Paciorek, et al.) discloses a smooth-surfaced layer of soft vinyl plastisol containing a fragrance coated on a base and protectively covered with a smooth flexible sheet having law vapor transmission. Out of this inventive composite, the layer having the volatile fragrance therein is comprised of plasticized vinyl resin.

U.S. Pat. No. 3,688,985 (Engel) discloses a plastic article impregnated with volatile matter. The article is made by treating a preformed synthetic water insoluble resin with a fragrance mixture. The mixture that the resin is exposed to preferably comprises an essential oil and surfactant emulsion.

U.S. Pat. No. 5,871,765 (Johnson, et al.) discloses a controlled release non-aqueous air care gel comprising diblock, triblock, multiblock and/or radial block copolymers based on synthetic thermoplastic rubbers.

U.S. Pat. No. 3,725,311 (Grubb) discloses an extrudable composition comprising mostly polyvinyl alcohol and acetate polymer and a volatile odor-neutralizing or modifying agent. The composition is extrudable at temperatures less than 250° F. in order to avoid flashing off the volatile fragrance compounds.

U.S. Pat. No. 6,123,906 (Farmer) discloses a plastic clip for scenting car interiors that is comprised of scented polypropylene.

U.S. Pat. No. 7,754,198 (Whitehead, et al.) discloses a translucent-to-clear plasticized polymeric matrix gel composition comprising a high loading of fragrance and a plastisol having a particular range of Shore hardness.

U.S. Pat. Nos. RE 40,941 and 6,316,520 (Hekal) discloses a solid fragrancing article comprised of a polymer with a hydrophilic agent dispersed therein. The product is solidified so that the hydrophilic agent forms passages in the product through which a desired composition can be released through to the surrounding environment. The polymer is preferably polyglycols poly(ethylene glycol), poly(propylene glycol), EVOH, pentaerithritol, PVOH, polyvinylpyrollidine, vinylpyrollidone or poly(N-methyl pyrollidone), or saccharide based compounds, glucose, fructose and their alcohols, mannitol, dextrin, and/or hydrolized starch.

U.S. Pat. No. 7,159,792 (Wheatley, et al.) discloses an air freshener that includes a scent material carried by and dispersible through a coherent, flexible and resilient polymer carrier material. The carrier material is a member selected from the group consisting of polyurethane, polyacrylate, polybutadiene, ethylene propylene elastomer, silicone, natural rubber, synthetic rubber, styrene/butadiene block copolymer, polyvinylchloride, ethylene vinyl acetate, polypropylene, ethylene/methacrylic acid copolymer, and mixtures thereof.

U.S. Pat. Nos. 6,838,033 and 6,309,715 (Lindauer, et al.) discloses a fragrant article comprising a polymer matrix, a fragrance, and a decorative object embedded therein. The polymer is preferably comprised of siloxanes, silicones, acrylics, carbonates, or polyesters.

U.S. Pat. No. 6,730,311 (Maleeny, et al.) discloses a scented picture frame assembly wherein a frame panel comprises a sustained-release fragrance material. The sustained-release fragrance material is a polyurethane/urea matrix, prepared by a process of reacting a urethane prepolymer with an aromatic diamine chain extender in the presence of a fragrance agent, wherein the aromatic diamine chain extender is selected from the group consisting of 4,4′-methylene-bis(2-chloroaniline), 4,4′-methylene-bis(3-chloro-2,6-diethylaniline), 4,4′-methylene-bis-aniline, diethyltoluenediamine, 5-t-butyl-2,4-toluenediamine, 3-t-butyl-2,6-toluenediamine, 5-t-amyl-2,4-toluenediamine, 3-t-amyl-2,6-toluenediamine, chlorotoluenediamine, and mixtures thereof.

U.S. Pat. No. 5,569,683 (Bootman, et al.) discloses a gel including a multi-component scented mixture disposed in a polymer matrix comprising the polymerization product of one or more ethylenically unsaturated monomers. Some of the preferred monomers for use in forming the polymer matrix include mono-functional ethylenically unsaturated monomers such as acrylamide, acrylic acid, hydroxyethylmethacrylate (HEMA), 2-ethoxyethyl acrylate, 2-phenoxyethyl acrylate, 2-(2-ethoxyethoxy) ethyl acrylate, t-butyl acrylamide, t-butyl acrylate, n-butyl acrylate, n-butyl acrylamide, 2-acrylamido-2-methylpropane sulfonic acid (AMPS) and the sodium salt thereof (NaAMPS), lauryl acrylate, lauryl methacrylate, stearyl acrylate, stearyl methacrylate, propylene glycol monoacrylate, caprolactone acrylate, nonylphenol acrylate, hexyl acrylate, isooctyl acrylate, carboxyethyl acrylate, isobornyl acrylate, polyether acrylate, nonylphenyl acrylate, ethoxylated nonylphenyl acrylate, and combinations thereof.

Lastly, U.S. Pat. No. 5,780,527 (O'Leary) discloses a gel element resulting from the in situ cross-linking of a functionalized polymer or copolymer with a cross-linking agent, in the presence of a perfume, deodorant or sanitizer. Preferred polymers include derivatives of butadiene, isoprene or chloroprene, such as for example maleinised polybutadiene of MW 5000 to 20,000, or maleinised polyisoprene of MW 200,000-500,000.

Lastly, some of the more relevant art related to thickened fragrance oils is as follows:

U.S. Pat. No. 7,584,901 (Boden, et al.) discloses a vapor dispensing system wherein the fragrance is preferably a thickened fragrance. The disclosure suggests that the fragrance may be thickened with Versagel®, Cab-O-Sil® or Tween®. There is no discussion as to a preferred viscosity for the thickened fragrance for use in the disclosed device.

PCT Application Publication WO 03/074095 (Maat) discloses a perfume gel composition comprising from 3 to 80 wt. % fragrance wherein the matrix is an anhydrous and substantially thermally reversible gel. The gelling agent is preferably a cellulose ester such as cellulose acetate propionate (CAP) and/or butyrate (CAB).

Also, U.S. Pat. No. 5,071,704 (Fischel-Ghodsian) discloses a laminated structure for the controlled release of vapors and scents. One of the inner layers of the laminate, called the reservoir layer, may be a thickened fragrance comprised of fragrance oil and hydroxypropyl cellulose.

In spite of decades of research in the field of water-based gels, non-aqueous fragranced polymers, and thickened fragrance oil systems, there is always room for new discoveries in aqueous or non-aqueous air freshening systems. Little is known regarding thickened fragrances in non-aqueous systems.

### SUMMARY OF THE INVENTION

In a preferred embodiment of the present invention, it has now been surprising discovered that the addition of small amounts of a cellulose derivative to fragrance oil generates a thixotropic mixture.

In another preferred embodiment of the present invention, small amounts of hydroxypropyl cellulose convert fragrance oil into a structured material having high yield values for free-standing applications or for coatings application on inanimate structures such as inorganic crystals or granules.

In another preferred embodiment of the present invention, from about 1.0 wt. % to about 10 wt. % of hydroxypropyl cellulose converts a fragrance oil into a rigid structure having a minimum yield stress of about 400 Pascal with no movement horizontally or vertically when inverted.

In another preferred embodiment of the present invention, a pseudoplastic gel comprising at least 4.0 wt. % hydroxypropyl cellulose in a fragrance oil finds use as a scented coating for crystals and other inanimate objects. The pseudoplastic characteristics allow the transfer of the scented gel to the object to coat the objects, with at least 80% structure recovery after the exposure to shear has ceased.

### BRIEF DESCRIPTION OF THE DRAWINGS

FIG. 1 is a plot of Stress, (Pa), versus the weight percent (wt. %) hydroxypropyl cellulose in a test fragrance oil.

FIG. 2 is an Anton Parr storage modulus and loss modulus plot for a gelled fragrance oil comprising 5 wt. % Klucel.

FIG. 3 is an Anton Parr storage modulus and loss modulus plot for a gelled fragrance oil comprising 4 wt. % Klucel.

FIG. 4 is an Anton Parr storage modulus and loss modulus plot for a gelled fragrance oil comprising 3 wt. % Klucel.

FIG. 5 is an Anton Pan storage modulus and loss modulus plot for a gelled fragrance oil comprising 2 wt. % Klucel.

### DETAILED DESCRIPTION OF THE INVENTION

The following description is of exemplary embodiments only and is not intended to limit the scope, applicability or configuration of the invention in any way. Rather, the following description provides a convenient illustration for implementing exemplary embodiments of the invention. Various changes may be made in the function of the elements described without departing from the scope of the invention as set forth in the appended claims. Changes in shape and size of the overall gelled fragrance oil do not depart from the intended scope of the invention.

That being said, the compositions of the present invention minimally comprise fragrance oil and a nonionic cellulose derivative. In a preferred embodiment, the nonionic cellulose derivative comprises hydroxyalkyl cellulose or alkyl hydroxyalkyl cellulose. In the more preferred embodiment, the cellulose derivative is hydroxypropyl cellulose (HPC). The non-aqueous gelled fragrance oil may also include synthetic polyacrylate or polyacrylamide polymers, dyes and colorants, and preservatives.

The present invention also comprises a method of manufacturing a non-aqueous fragrance gel comprising fragrance oil and a cellulose derivative.

The present invention also comprises a method of coating an object, such as a salt crystal or an inorganic granule, with a pseudoplastic gel comprising fragrance oil and hydroxypropyl cellulose.

Nonionic Cellulose Derivative

The present invention necessarily comprises a nonionic cellulose derivative. Addition of a very specific and unique nonionic cellulose derivative to non-aqueous fragrance oil unexpectedly forms a pseudoplastic gel. In particular, the combination of fragrance oil and hydroxypropyl cellulose forms a self-supporting gel.

Useful nonionic cellulose derivatives include hydroxyethyl cellulose (HEC), hydroxypropyl cellulose (HPC), ethylhydroxyethyl cellulose (EHEC), hydroxypropylhydroxyethyl cellulose (HPHEC), methyl cellulose (MC), methylhydroxypropyl cellulose (MHPC), methylhydroxyethyl cellulose (MHEC), hydrophobically modified hydroxyethyl cellulose (HMHEC), hydrophobically modified hydroxypropyl cellulose (HMHPC), hydrophobically modified ethylhydroxyethyl cellulose (HMFEHEC), hydrophobically modified hydroxypropylhydroxyethyl cellulose (HMHPHEC), hydrophobically modified methyl cellulose (HMMC), hydrophobically modified methylhydroxypropyl cellulose (HMMHPC), hydrophobically modified methylhydroxyethyl cellulose (HMMHEC), and mixtures thereof. These substances are available as Klucel®; Ashland, Methocel®, Cellosize®; Amerchol; Natrosol®; Hercules and Blanose®; Aqualon, Aquasorb®, Ambergum®; Hercules; Cellgon®; Montello. More preferred is hydroxypropyl cellulose (HPC), available under the Klucel® brand from Ashland. The nonionic cellulose derivative is incorporated into the fragrance oil to form the gel at from about 1 wt. % to about 10 wt. % actives, and most preferably from about 4.0 wt % to about 10 wt. %, based on the total weight of the gel composition.

Fragrance Oil

The rigid and pseudoplastic non-aqueous fragrance gel of the present invention necessarily includes fragrance oil. Fragrance oil in accordance with the present invention may comprise one of more volatile organic compounds available from any of the now known, or hereafter established, perfumery suppliers, such as International Flavors and Fragrances (IFF) of New Jersey, Givaudan of New Jersey, Firmenich of New Jersey, etc. Many types of fragrances can be used in the present invention. Preferably the fragrance materials are volatile essential oils. The fragrances, however, may be synthetically derived materials (aldehydes, ketones, esters, etc.), naturally derived oils, or mixtures thereof. Naturally derived fragrance substances include, but are not limited to, musk, civet, ambergis, castoreum and like animal perfumes; abies oil, ajowan oil, almond oil, ambrette seed absolute, angelic root oil, anise oil, basil oil, bay oil, benzoin resinoid, bergamot oil, birch oil, bois de rose oil, broom abs., cajeput oil, cananga oil, capsicum oil, caraway oil, cardamon oil, carrot seed oil, cassia oil, cedar leaf, cedarwood oil, celery seed oil, cinnamon bark oil, citronella oil, clary sage oil, clove oil, cognac oil, coriander oil, cubeb oil, cumin oil, camphor oil, dill oil, estragon oil, eucalyptus oil, fennel sweet oil, galbanum res., garlic oil, geranium oil, ginger oil, grapefruit oil, hop oil, hyacinth abs., jasmin abs., juniper berry oil, labdanum res., lavander oil, laurel leaf oil, lavender oil, lemon oil, lemongrass oil, lime oil, lovage oil, mace oil, mandarin oil, mimosa abs., myrrh abs., mustard oil, narcissus abs., neroli bigarade oil, nutmeg oil, oakmoss abs., olibanum res., onion oil, opoponax res., orange oil, orange flower oil, origanum, orris concrete, pepper oil, peppermint oil, peru balsam, petitgrain oil, pine needle oil, rose abs., rose oil, rosemary oil, sandalwood oil, sage oil, spearmint oil, styrax oil, thyme oil, tolu balsam, tonka beans abs., tuberose abs., turpentine oil, vanilla beans abs., vetiver oil, violet leaf abs., ylang ylang oil and like vegetable oils, etc. Synthetic fragrance materials include but are not limited to pinene, limonene and like hydrocarbons; 3,3,5-trimethylcyclohexanol, linalool, geraniol, nerol, citronellol, menthol, borneol, borneyl methoxy cyclohexanol, benzyl alcohol, anise alcohol, cinnamyl alcohol, β-phenyl ethyl alcohol, cis-3-hexenol, terpineol and like alcohols; anethole, musk xylol, isoeugenol, methyl eugenol and like phenols; a-amylcinnamic aldehyde, anisaldehyde, n-butyl aldehyde, cumin aldehyde, cyclamen aldehyde, decanal, isobutyl aldehyde, hexyl aldehyde, heptyl aldehyde, n-nonyl aldehyde, nonadienol, citral, citronellal, hydroxycitronellal, benzaldehyde, methyl nonyl acetaldehyde, cinnamic aldehyde, dodecanol, α-hyxylcinnamic aldehyde, undecenal, heliotropin, vanillin, ethyl vanillin and like aldehydes; methyl amyl ketone, methyl β-naphthyl ketone, methyl nonyl ketone, musk ketone, diacetyl, acetyl propionyl, acetyl butyryl, carvone, menthone, camphor, acetophenone, p-methyl acetophenone, ionone, methyl ionone and like ketones; amyl butyrolactone, diphenyl oxide, methyl phenyl glycidate, gamma.-nonyl lactone, coumarin, cineole, ethyl methyl phenyl glicydate and like lactones or oxides; methyl formate, isopropyl formate, linalyl formate, ethyl acetate, octyl acetate, methyl acetate, benzyl acetate, cinnamyl acetate, butyl propionate, isoamyl acetate, isopropyl isobutyrate, geranyl isovalerate, allyl capronate, butyl heptylate, octyl caprylate octyl, methyl heptynecarboxylate, methine octynecarboxylate, isoacyl caprylate, methyl laurate, ethyl myristate, methyl myristate, ethyl benzoate, benzyl benzoate, methylcarbinylphenyl acetate, isobutyl phenylacetate, methyl cinnamate, cinnamyl cinnamate, methyl salicylate, ethyl anisate, methyl anthranilate, ethyl pyruvate, ethyl α-butyl butylate, benzyl propionate, butyl acetate, butyl butyrate, p-tert-butylcyclohexyl acetate, cedryl acetate, citronellyl acetate, citronellyl formate, p-cresyl acetate, ethyl butyrate, ethyl caproate, ethyl cinnamate, ethyl phenylacetate, ethylene brassylate, geranyl acetate, geranyl formate, isoamyl salicylate, isoamyl isovalerate, isobornyl acetate, linalyl acetate, methyl anthranilate, methyl dihydrojasmonate, nopyl acetate, β-phenylethyl acetate, trichloromethylphenyl carbinyl acetate, terpinyl acetate, vetiveryl acetate and like esters, and the like. Suitable fragrance mixtures may produce an infinite number of overall fragrance type perceptions including but not limited to, fruity, musk, floral, herbaceous, edible, and woody, or perceptions that are in-between (fruity-floral for example). Typically these fragrance mixtures are compounded by the fragrance houses by mixing a variety of these active fragrance materials along with various solvents to adjust cost, evaporation rates, hedonics and intensity of perception. Well known in the fragrance industry is to dilute essential fragrance oil blends (natural and/or synthetic) with solvents such as ethanol, isopropanol, hydrocarbons, acetone, glycols, glycol ethers, water, and combinations thereof, to make the purchased “fragrance oil” raw material blend more easily handled by the formulator and to adjust the rate of evaporation of the volatiles and the hedonics. The preferred fragrance oil for use in the gel air freshener of the present invention may be comprised of a mixture of many fragrance actives and volatile solvents, sometimes along with smaller amounts of emulsifiers, stabilizers, wetting agents and preservatives. More often than not, the compositions of the fragrance oil purchased from the various fragrance supply houses remain proprietary and thus can only be described in general terms.

The fragrance material is preferably incorporated at a level of from about 90% to about 99% by weight in the gel, based on the total weight of the finished fragrance gel.

Optional Ingredients

Fragrance gels may also include additional ingredients to increase the stability of the solidified gel. Such materials include, but are not limited to: various natural gums derived from non-marine biological sources like gum Arabic, gum ghatti, gum tragacanth, Karaya gum, Guar gum, Locust Bean gum, beta-glucan, Chicle gum, Dammar gum, glucomannan, Mastic gum, Spruce gum, Tara gum, Cassia gum, Gellan gum, and xanthan gum, and mixtures thereof; and various small molecular weight salts like sodium chloride, potassium chloride, magnesium chloride, calcium chloride, sodium acetate, potassium acetate, magnesium acetate, calcium acetate, sodium sulfate, potassium sulfate, magnesium sulfate, and calcium sulfate. To further stabilize the gel of the present invention, the total amount of these optional compounds in the finished gel may be from about 0.01 wt. % to about 10 wt. %, based on the total weight of the air freshener gel composition.

The gel in accordance with the present invention may also include dyes, pigments or other suitable colorants to provide aesthetic appeal to the retail gel air freshener product. Such dyes may include FD&C and/or D&C Yellows, Reds, Blues, Greens and Violets, or really any other dye or pigment, and such raw materials are commonly purchased in either powder or liquid form from numerous suppliers. Dyes and/or pigments are incorporated at levels sufficient to provide light color to deep color to the gelled fragrance product. When the optional dyes or pigments are incorporated to produce a colored air freshener gel, they are added at from about 0.0001% to about 1% by weight, depending on the concentration of the colorants (e.g. if a liquid/diluted dye or a neat powder is the raw material). The dyes and/or pigments will either dissolve or disperse to lend a visible color to the non-aqueous fragrance gel.

The gel in accordance with the present invention may also comprise one or more preservatives to help prevent dye fading and/or mold or other microbial growth in and/or on the gel. The preferred microbial preservatives include Neolone® and Kathon® products from Lonza and Rohm & Haas. These materials are incorporated at the manufacturers' recommended levels in the air freshener gel to discourage bacterial and mold growth. An ultraviolet inhibitor and/or an antioxidant such as BHT may also be added to the air freshener gel to reduce dye fading that may become an issue when the air freshener is opened and exposed to light by the consumer.

The gel in accordance with the present invention is categorized as non-aqueous. As such, and what is meant herein, is that the gelled fragrance oil is substantially free of water. There is of course the possibility that small amounts of water are entrained into the gelled fragrance by way of the fragrance oil blend. As mentioned above, some fragrance suppliers may have small amounts of solvents, including water, in their commercialized fragrance oils. The amount of water, if any, in these fragrance oils will typically be less than 1% by weight of the fragrance oil composition.

Method of Manufacturing Non-Aqueous Fragrance Gels of the Present Invention

The method of manufacturing a non-aqueous viscous gel having pseudoplastic rheology comprises the steps of (1) placing a suitable fragrance oil into a vessel equipped with mechanical stirring, and (2) adding in slowly a nonionic cellulose derivative in an amount suitable for gelling at the desired rheology endpoint. The mechanical stirring may be anything from simple stirring using an immersed paddle to a homogenizer. The nonionic cellulose derivative, such as a hydroxyalkyl cellulose and most particularly hydroxypropyl cellulose, is preferably added as a neat powder. The cellulose derivative may be added as a solution or dispersion in a suitable solvent. Such a solvent may be one of the solvents already present as a diluent in the fragrance oil. Since the resulting gel is pseudoplastic, cessation of mechanical stirring will result in restoration of the rigid structure. However, the gel may be reversibly sheared, and mechanical agitation will enable the gel to be transferred from a mixing vessel to some other vessel for a subsequent manufacturing step or packaging. For example, mechanical forces allow for the transfer of the finished pseudoplastic gel from the mixing vessel to a tumbler where the gel may be used to coat objects such as salt granules or crystals.

Use of the Non-Aqueous Pseudoplastic Gel as a Coating.

As mentioned above, the pseudoplastic gel of the present invention is ideally suited for use as a coating. The advantage of this particular coating is its rigidity, meaning that once it is coated onto an object, it will not move off. Objects that may be coated with the non-aqueous fragrance gel of the present invention include, but are not limited to, potpourri, compressed pellets and tablets, beads, stones, sticks, sand, silica, fabric swatches, and/or crystals. Crystals that may be coated with the gelled fragrance oil of the present invention include any inorganic or organic substance, such as sodium chloride, potassium chloride, sucrose, copper sulfate, borax, etc. The size of the material to be coated by the present inventive gel is infinite. The size may range from the tiniest grains like sand grains up to large baseball-size objects such as large wooded balls. It is most preferred to use the non-aqueous fragrance gel of the present invention to coat salt or sugar crystals. These preferred crystals may range in size from about ⅛ inch to about 1 inch. The result of coating such crystals is a salt or sugar crystal air freshener that has optical clarity and beauty.

Results and Discussion

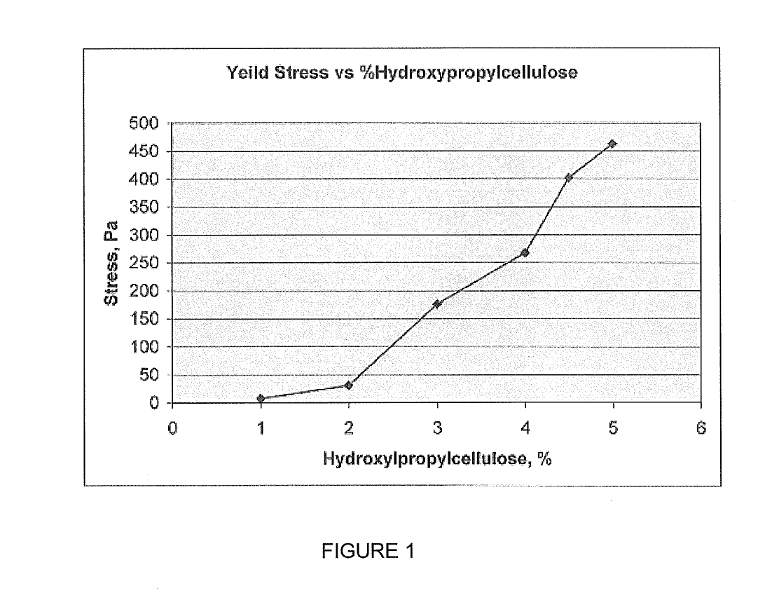

TABLE 1 delineates several non-limiting embodiments of the present invention. Gel A in TABLE 1 shows remarkable pseudoplastic rheology. FIG. 1 shows the Yield Stress in Pascal versus various levels of hydroxypropyl cellulose.

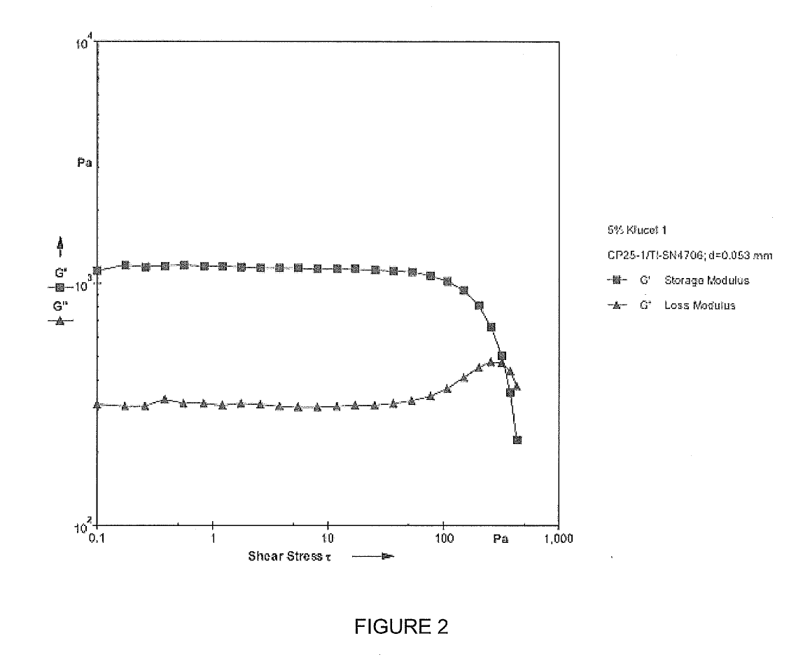

In general, about 4 wt. % or greater of hydroxypropyl cellulose produces a self-standing gel. What is meant herein by “self-standing” is that if the gel is placed on a flat surface, that surface can be lifted to 90° from horizontal and the gel does not move horizontally. The surface can also be completely inverted and the gel will not flow. It is hence, self-supporting, self-standing and rigid. The crossover point (where the loss modulus G″ and storage modulus G′ curves intersect) is a good measure of the stability of the gel and if it is rigid. FIGS. 2-5 show plots of loss modulus and storage modulus measured by Anton Paar rheometer, and it is evident that increasing amounts of cellulose derivative has a dramatic effect on rigidity of the pseudoplastic gel.

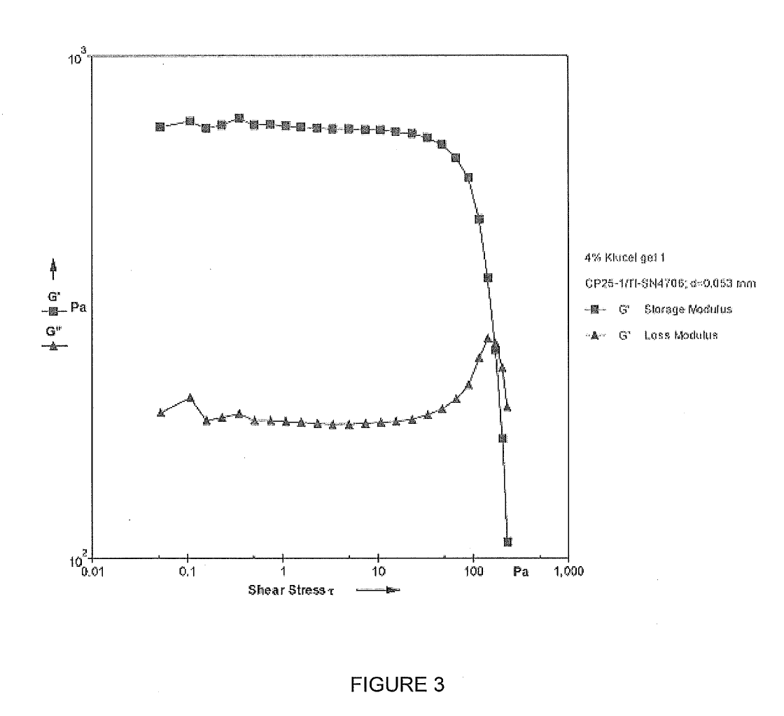

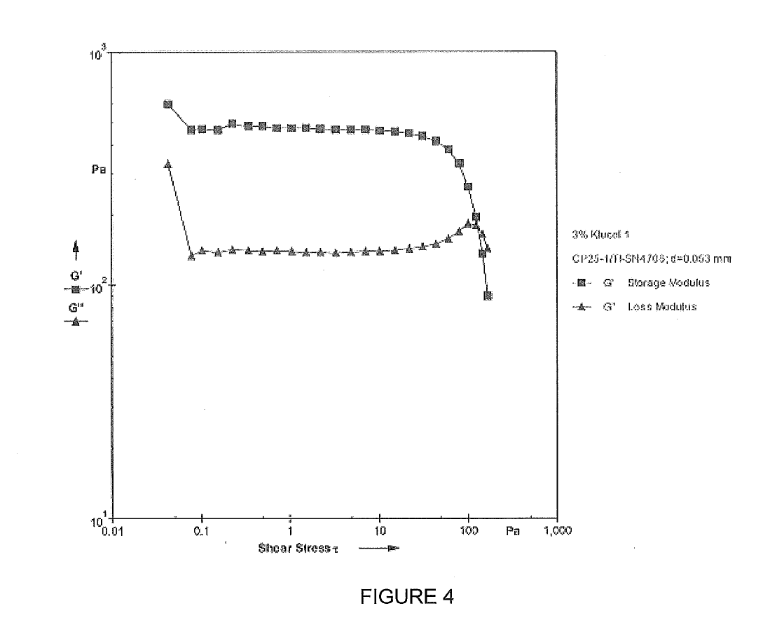

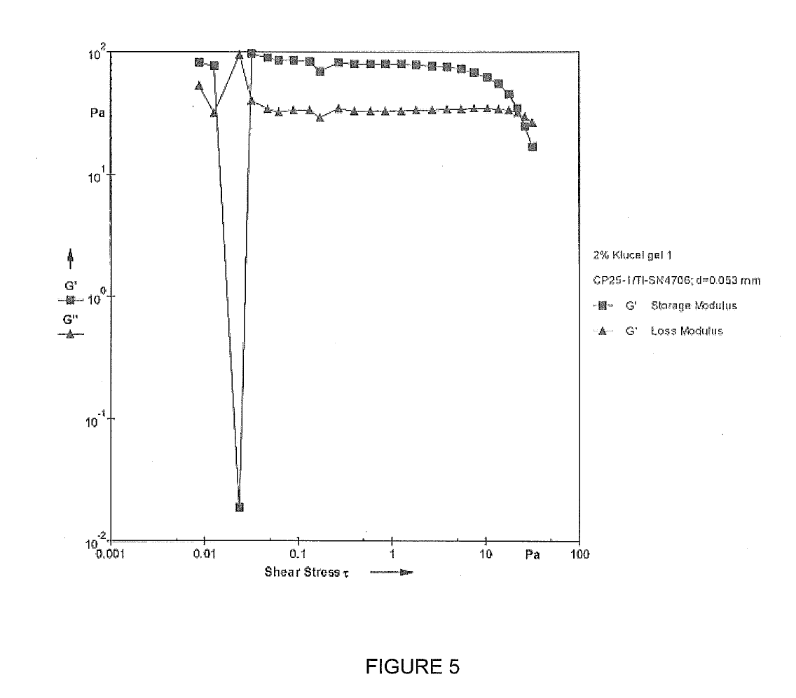

TABLE 1
Non-Aqueous Fragrance Oil Gels

| Ingredients | | | | | | |
|---|---|---|---|---|---|---|
| (wt. % actives) | A | B | C | D | E | F |
| Hydroxypropyl | 4.5 | 1.0 | 2.0 | 3.0 | 4.0 | 5.0 |
| cellulose | | | | | | |
| (Klucel ®) | | | | | | |
| Fragrance Oil | 95.5 | 99.0 | 98.0 | 97.0 | 96.0 | 95.0 |
| (various) | | | | | | |
| Dyes, | Present | Present | Present | Present | Present | Present |
| pigments, | | | | | | |
| preservatives | | | | | | |
| Total | 100 | 100 | 100 | 100 | 100 | 100 |
| Rheology | | | | | | |
| Yield Stress | 400 | Near 0 | <50 | 150-200 | 250-300 | >450 |
| (Pascal) | | | | | | |
| Crossover | | | FIG. 5 | FIG. 4 | FIG. 3 | FIG. 2 |
| (intersection | | | | | | |
| of G′ G″) | | | | | | |

As evident from the disclosure herein, hydroxypropyl cellulose uniquely thickens fragrance oil into a rigid gel having pseudoplastic rheology. Such gelled fragrance oil finds use as a coating to make scented potpourri and scented crystal air fresheners. The gel also finds use as an air freshener in and of itself simply by placing the non-aqueous gel in a suitable container and exposing the gel to ambient air. The container may feature a grating or some other structure to prevent handling of the gel but still allow the fragrances to permeate through.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

We claim:

1. A rigid non-aqueous gel comprising fragrance oil and a hydroxyalkyl cellulose.

2. The gel of claim 1, wherein said hydroxyalkyl cellulose is hydroxypropyl cellulose.

3. A method of manufacturing a non-aqueous psuedoplastic fragrance gel, said method comprising the steps of:
a) placing a suitable fragrance oil at ambient conditions in a vessel equipped with mechanical agitation; and
b) adding between about 1.0 wt. % and 10.0 wt. % of a hydroxyalkyl cellulose to the agitated fragrance.

4. The method of claim 3, wherein said mechanical agitation is stirring from a paddle blade mixer.

5. The method of claim 3, wherein said mechanical agitation is homogenization from a suitable homogenizer.

6. The method of claim 3, wherein said hydroxyalkyl cellulose is dry powered hydroxypropyl cellulose.

7. A method of coating a pellet, tablet, crystal, and/or inanimate objects with a non-aqueous pseudoplastic fragrance gel, said method comprising the steps of:
a) placing a suitable fragrance oil at ambient conditions in a vessel equipped with mechanical agitation;
b) adding between about 1.0 wt. % and 10.0 wt. % of dry powdered hydroxypropyl cellulose to the agitated fragrance to form a pseudoplastic fragrance gel;
c) transferring said pseudoplastic fragrance gel to a tumble mixer containing said pellets, tablets, crystals, and/or inanimate objects; and
d) tumbling said pellets, tablets, crystals, and/or inanimate objects with said pseudoplastic fragrance gel until a suitable level of coating of the pellets, tablets, crystals, and/or inanimate objects is achieved.
